(19) United States
(12) Patent Application Publication
(10) Pub. No.: US 2004/0106951 A1
(43) Pub. Date: Jun. 3, 2004

(54) Use of electric fields to minimize rejection of implanted devices and materials

(76) Inventors: Carl Frederick Edman, San Diego, CA (US); Darrel Dean Drinan, San Diego, CA (US)

Correspondence Address: Carl Edman, Suite 152, 11772 Sorrento Valley Road, San Diego, CA, 92121, US

(21) Appl. No.: 10/722,306

(22) Filed: Nov. 24, 2003

Related U.S. Application Data
(60) Provisional application No. 60/428,583, filed on Nov. 22, 2002.

(51) Int. Cl. A61N 1/00

(52) U.S. Cl. 607/2

(57) ABSTRACT

This invention relates to the electric field method to reduce fibrous capsule formation adjacent to the surface of implanted medical devices and associated apparatus for generating electrical currents to reduce fibrous capsule formation. The invention has utility with medical devices or systems such as those providing long term parenteral drug delivery, fluid infusion or analyte sampling/measurement. The apparatus of the invention may be constructed as a part of the medical device or may be constructed of separate elements while providing the benefit of the electric fields to the medical device.

### CROSS-REFERENCE TO RELATED APPLICATIONS

This application claims the benefit of U.S. Provisional Application Ser. No. 60/428,583, filed Nov. 22, 2002, which is incorporated herein by reference.

### FIELD OF THE INVENTION

This invention relates to the use of electric fields to reduce fibrous capsule formation adjacent to the surface of implanted medical devices. Reduced capsule formation facilitates the transfer of fluids into or out of an interior lumen of an implanted device and the surrounding tissue or between the surface of an implanted device and surrounding tissue. Medical devices employing such electric fields may be part of a drug delivery system/device or a biofluid sampling system/device intended for use within a mammalian body. The invention provides for methods and apparatus to supply electric currents to generate such fields as either stand alone apparatus or as part of larger medical devices or systems.

### BACKGROUND OF THE INVENTION

Limiting the lifetime of devices implanted within the body of a mammalian subject is the body's rejection reaction-to these materials, termed the “foreign body response”. In this context, the foreign body response consists any or all of those events initiated by the body in reaction to introduced material. This includes, but is not limited to, inflammation response, migration of macrophages or other wound/repair cells to the location, altered cell type of the surrounding tissue, deposition of fibrous proteins and related materials not normally observed within the particular tissue in those forms or levels, and the walling off or encapsulation of the device by the body by a fibrous capsule.

For those devices which can support this rejection response, i.e. those devices which can reside in a fully functional state when encapsulated, the body's foreign body response is not as hindering as for those devices, i.e. catheters, ports, or other fluid transfer points, which require as part of their function, minimal obstruction of the passage of fluids or other materials into or out of an interior or lumenal space within a device and the surrounding tissue. However, in both classes of devices, minimization of the rejection response may be a means to possibly extend device useful lifetime within the body.

To date this minimization has been accomplished by use of composition of the materials in contact with the body, selective coatings and/or modifications of the surface of devices to promote acceptance by the body. For instance, Joseph and Torjman (U.S. Pat. No. 6,471,689) teach the use of bilayer membranes to encourage neovascularization and minimize capsule formation. Such passive means to control the rejection response by themselves have not always proved effective. In contrast, use of an active means, that of controlled local electric fields, to mediate the body's foreign body response, has not been adopted.

In considering the body's reaction to introduced foreign materials and substances, the cascade of events is considered to follow the biological events associated with a wound healing response. Applied electric fields, i.e. electrical currents, have been employed successfully to accelerate the processes associated with wound healing. Miller (U.S. Pat. No. 4,846,181) teaches the use of pulsed electrical stimulation of varying polarities throughout the treatment cycle to enhance soft tissue wound healing. A variety of techniques and electrical currents are available as means to accelerate or enable both soft tissue and bone healing processes. (See, for example, Chapter 21, Sussman, C and Byl, N N, “Electrical Stimulation for Wound Healing” in Wound Care, 2^{nd} Edition, Sussman C and Bates-Jensen, B M, editors, Aspen Publishers, Gaithersburg, Md. 2001, and references cited therein.) To date however, the use of electrical currents to retard or diminish wound healing response (and by implication, subsequent fibrous capsule formation) has not been described.

Use of electric fields as a means of diminishing the wound healing response is supported by the work of J. D. Reich, et al. (J Amer Acad Derm 1991 ;25:40-6) whereby they demonstrate a twofold reduction in mast cell infiltrate in cutaneous wounds upon the periodic application of pulsatile electrical currents to the dermis.

In a different application of electrical forces, Rise (U.S. Pat. No. 5,853,424) teaches the use of static surface charges upon surfaces of implanted devices to retard or prevent tissue ingrowth in infusion catheters. Such charges are created using chemical means or by electrical means through the application of source of electrical potential. However, there remains a need for methods and apparatus to minimize the body encapsulation response about medical devices and systems located beneath the surface of the skin.

### SUMMARY OF THE INVENTION

This invention relates to the methods and apparatus for introducing electrical currents into the body for the purpose of minimizing fibrous capsule formation. Such methodology represents a novel and significant advancement in the art of medical devices, both in the area of drug delivery and in biofluid sampling for the purpose of determining analytes.

The method of this invention includes the application of electrical currents through subcutaneous tissue through at least one active first electrode positioned in close proximity to a critical feature of an implanted or subcutaneous medical device to at least one second electrode located elsewhere either on the device or otherwise enabling completion of the electrical circuit. It is the object of this invention that by use of these electrical currents, fibrous capsule formation in the vicinity of one or more critical features is reduced or eliminated thereby improving device performance and useful lifetime.

The apparatus of the invention includes at least one first electrode in fluidic contact with surrounding tissue, at least one second electrode in fluidic contact with surrounding tissue and the control circuitry plus power supply enabling the delivery of an electrical current through the tissue from at least one first electrode to at least one second electrode.

In a broader description of the method of this invention, the invention utilizes electrical currents which are substantially DC (direct current) in nature, as opposed to AC (alternating current) in nature. In one embodiment of the invention, these DC currents are pulsatile in form. Apparatus for the generation, control and delivery of such currents, whether pulsatile or constant, including the electrode, control circuitry and power, are contained within the broader aspects of the invention.

Embodiments of the method and apparatus of the invention also includes the use of structures, materials or electrical current protocols useful for diminishing possible deleterious effects resultant from the introduction of an electrical current into a fluid, e.g. electrolysis by-products. In one embodiment of the invention, the use of pulsatile currents is utilized to reduce possible deleterious effects. In yet other embodiments of the invention, the electrode surface is separated from contact with surrounding tissue using structures such as porous coatings, gels, sequestration within lumen of devices or by use of mesh-like screens. Such separation permits additional features, e.g. natural buffering capacity, to ameliorate possible deleterious effects resultant from the introduction of the electrical current.

One embodiment of the invention includes the use of the apparatus of the invention as part of a percutaneous therapeutic agent delivery system or device.

In an alternate embodiment of the invention, the apparatus of this invention is incorporated into a fully implanted system or device providing long term sampling for analytes within biofluids or drug delivery.

In yet other embodiments of the invention, the apparatus of the invention is composed of separable elements such that a part of the apparatus is incorporated into a medical device and a second part, e.g. a second electrode, is not.

This invention may be embodied in many different forms and should not be construed as being limited to the embodiments described above. In addition, various embodiments of the invention may combine one or more additional embodiments as part of the scope of the overall invention. Those skilled in the art will readily understand the basis and means of the invention as described by the embodiments.

### BRIEF DESCRIPTION OF THE DRAWINGS

FIG. 1—Model of the electrode/electrolyte interface depicted as a portion of an electric circuit.

FIG. 2—Illustration of one form of a semipermeable structure segregating an electrode from surrounding tissue. The semipermeable structure also serves as a site for fluid delivery into surrounding tissue.

FIG. 3—Diagram of one embodiment of control circuitry for the generation of pulsatile electrical currents.

FIG. 4—Diagram of one embodiment of a catheter-like device employing the apparatus of this invention having both first and second electrodes upon the device surface.

FIG. 5—Cross-sectional view of a portion of the embodiment shown in FIG. 4.

FIG. 6—Illustration of an alternate embodiment of a catheter-like device utilizing the apparatus of this invention. A first set of electrodes is incorporated into the structure of the device with the second electrode being independently positioned in adjacent tissue.

FIG. 7—Cross sectional diagram of one embodiment of a fully implantable device for the delivery of drugs and therapeutic agents also incorporating the apparatus of this invention.

### DEFINITIONS

1. Apparatus of this invention—The means to deliver controlled electrical currents through subdermal regions of the body. These means include, but are not limited to, at least one first electrode, at least one second electrode, an electrical power supply and control circuitry.

2. Biofluids—Fluids found in extracellular environments, e.g. interstitial fluid, cerebrospinal fluid, throughout the body of the subject which may contain a variety of materials, including but not limited to, proteins, hormones, nutrients, electrolytes, catabolic products, or introduced foreign substances.

3. Critical structures/features of devices—Those surfaces, structures or regions of a device which are in contact with the surrounding tissue and require either fluid passage between the device and surrounding tissue through this structure or having the need to access the surrounding tissue at the location of these surfaces, structures or regions, e.g. the location of an optical sensor, and where the presence of a fibrous encapsulation diminishes device performance.

4. Device—Device in the context of this invention refers to medical devices, instruments, systems, structures, materials or other objects non-native to the host body and may be inorganic or organic in composition which are placed either in part or in whole in one or more subdermal regions of a subject.

5. Electrophoresis—The movement of molecules or particles (possessing a net charge) under the influence of an electric field. This electric field results from the passage of an electric current through solution containing charged particles from one electrode to another.

6. Subdermal—Beneath the skin. In the context of this invention, this region may include, but is not limited to, tissues, organs, cavities, fluids, vascular or connective structures located within the body of a subject. In this context, subdermal also includes regions of the dermis that would be pierced or other penetrated by devices such as microdelivery needles.

7. Subject—A human or mammalian subject who has one or more devices employing the apparatus of this invention.

### DETAILED DESCRIPTION OF THE INVENTION

The invention generally relates to novel use of electric fields to diminish the body's rejection response to implanted drug delivery and biofluid sampling devices. The present invention specifically relates to the method of delivering electrical current and apparatus for the delivery of these currents, in a variety of forms, amplitudes or periodicities. The invention described herein provides for methods and devices enabling long term continuous or periodic monitoring of physiological conditions by subdermal systems or the administration of therapeutic agents by subdermal systems.

The primary elements associated with the invention is the location of one or more first electrodes in the vicinity of critical structures or features of the implanted medical device, the positioning of one or more second or counter electrodes in a region not critical to device performance, the activation of at least one critical structure electrode and at least one counter electrode completing a circuit and the subsequent mobilization of charged molecules and/or the direction of cell and cellular material associated with the body's rejection response or impaired device function from the critical locations upon passing an electrical current from at least one critical structure electrode to at least one counter electrode. In the context of this invention, structures or features of medical devices critical for device performance are those surfaces or regions of the device exposed to the surrounding tissue requiring either fluid passage between the device and the tissue or having the need to access the surrounding tissue, e.g. optically sense, and which the presence of a fibrous encapsulation diminishes optimal device performance.

We claim that by activating at least one first electrode in close proximity to a critical structure or feature of a subcutaneous device or material, e.g. a fluid infusion site, and having at least one second electrode apart from this location, charged signaling peptides, proteins, or certain cell types such as fibroblasts, macrophages, mast cells, etc., associated with a walling off, encapsulation, clogging or foreign body response to the device will be physically moved or directed from the critical structure, thereby limiting the extent to which the rejection processes initiated by these charged species will detrimentally affect the medical device.

As this electrophoretic technique is substantially different from the passive approaches previously employed to minimize rejection, e.g. surface modification, it may be employed by itself or in combination with one or more of these other techniques in order to minimize rejection and/or encapsulation of the medical device or portions thereof.

The basis for minimizing the rejection response and subsequent fibrous encapsulation is to employ electric fields as a novel active means to limit this activity. In particular, to use the technique of electrophoresis is employed for this purpose. Electrophoresis is well known to those in the art as the basis for several analytical tools in biochemistry and related disciplines. In this invention, electrophoresis is employed as a means for mobilizing or directing biomaterials (typically proteins and modified proteins, or certain cell types) associated with the rejection response away from critical device locations, surfaces and surrounding areas.

Electrophoresis is the movement of molecules or particles (possessing a net charge) under the influence of an electric field. This electric field results from the passage of an electric current through solution containing charged particles from one electrode to another. The forces underlying electrophoretic migration and mobility are well understood. This technique has been applied to devices and methods for the delivery of charged species to both intracellular and extracellular fluids. However, electrophoresis has not been used as means for limiting or preventing the body's foreign body response to introduced materials, structures or devices.

By preventing or diminishing the localization of these biomaterials, e.g. proteins, signaling peptides, or specific cell types associated with the wound healing cascade and/or foreign body response including fibrous capsule formation, in the vicinity of critical structures or features by the use of electrophoresis, the rejection response (encapsulation) or clogging will be restricted or eliminated entirely enabling longer useful lifetimes for implanted devices or materials. The method of electrophoresis and associated apparatus for delivery of electrical currents for the purpose of electrophoresis provides the basis of this invention.

Typically, proteins and molecules such as signaling peptides possess a net charge at physiological pH (pH 7.0-7.5). Signaling peptides or molecules in this context refer to those molecular entities which initiate a cascade of events, e.g. acting as chemo-attractants causing cell migration to vicinity of the device or the triggered deposition of collagens or other materials associated with rejection, etc. The novel use of electric fields, i.e. electrophoresis, mobilizes all or a portion of these charged biomolecules associated with clogging or impaired function away from those critical device locations which govern either device performance or overall device lifetime within the body. More precisely, electric fields are employed to mobilize charged molecules/cell types that either by themselves would build up and occlude critical features or which are associated with the rejection response of the body towards foreign objects.

In general, the rate of migration depends upon the applied electric field, and is inversely proportional to the viscosity of the solution. It should be further noted that the migration of proteins and peptides (or other charged biomolecules associated with the rejection response) is also dependent of a number of factors beyond the applied electric field and solution viscosity. In particular, the composition of the biomolecule, e.g. the amino acids or other charged moieties and their location within the three dimensional structure, determines the biomolecule's net charge at a given local pH as well as the biomolecule's overall size and shape. That is, electrophoretic mobility of a biomolecule is also proportional to the biomolecule's net charge and inversely proportional to the its size (and shape).

In addition to the above considerations, the ion composition of the surrounding medium determines the effect of the electric field upon the biomolecule in question. That is, in low ionic strength medium, movement of a charged molecule, e.g. a negatively charged protein, results in a separation from its counter ions, (typically Na+ ions). In this circumstance, charge separation forces tend to counteract electrophoretic migration of the biomolecule thus hindering its migration through the solution. In contrast, in high ionic strength mediums, such as interstitial fluid, numerous counterions, typically cations such as Na+, are present. In this environment, migration of the biomolecule is not be hindered by loss of counter ions since large quantities are present in solution. However, the presence of such large quantities of counter ions results in an ion “cloud” shielding the biomolecule from the electric field, thereby reducing the force upon the biomolecule and hence its observed velocity. In certain instances, the ionic species themselves, e.g. Mg++ or Ca++, may govern or influence the motility of cell types associated, either directly or indirectly, with capsule formation. Thus, the electrical current may influence the nature and relative quantities of signaling molecules, peptides, ions and other mediators of the wound healing cascade and ultimately of fibrous capsule formation.

Alternatively, the application of electrical currents through the tissue may directly influence the motility of certain cell types. That is, the orientation and strength of electric fields generated by the passage of an electrical current have been shown to guide the migration of select cell types such as endothelial cells and fibroblasts in vitro and in vivo. Fibroblasts in particular have been shown to migrate towards the cathode under the influence of an applied current. Fibroblasts are also considered responsible for the generation of the bulk of the collagen forming the fibrous capsule surrounding implants.

Thus, to minimize the infiltration of fibroblasts in the vicinity of critical device features, in one embodiment of the invention, one or more first electrodes in close proximity to critical features of a device serves as anodes (positive bias) with one or more second electrodes (counter electrodes) serving as the cathode (negative bias). Overall, the actions of the applied electrical current may either affect certain key signaling molecules in the wound healing cascade directly or the electric fields engendered by the passage of this current may influence the motility of cells involved in the wound healing cascade. The exact form, amplitude and polarity of the currents applied are determined by the tissue/cell types involved and the functional requirements of the medical device and the scope of this invention is not limited to any one embodiment of these.

The process of running an electric current through a solution may result in several possibly detrimental side effects, dependent upon the nature and extent of applied current and the dimensions/types of electroactive surfaces, e.g. electrodes, employed. Chief among these effects are: the generation of acid and base at the anode and cathode (respectively); the highly reactive electrolysis zone immediately adjacent to the electrode surface; and the possible formation of gas bubbles at the electrodes. The ability to deal with these potentially detrimental effects represents additional novel and unobvious aspects to the invention described herein.

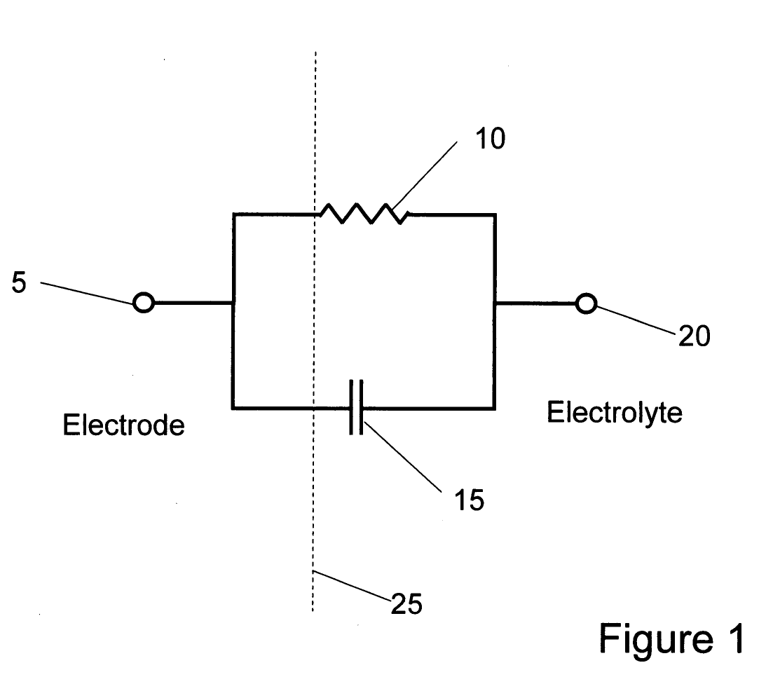

The introduction of an electrical current at an electrode/electrolyte interface is typically modeled as a capacitor in parallel to a resistance (FIG. 1). In this model, the boundary between the electrode (5) and the surrounding fluid or electrolyte (20) is represented by the dashed line (25). The capacitor (15) represents the capacitance of the double layer of electrolytes formed at the boundary (25) and the resistance (10) represents the faradaic reaction(s) of chemical species at the electrode surface. Based upon this simple model, one effective means to reduce or minimize possibly detrimental activities in to introduce the current in a pulsatile fashion, analogous to the passage of high frequency electrical signals through capacitors. By doing such, the faradaic reactions are minimized, lessening the generation of the deleterious agents.

Pulsatile currents are typically characterized by the pulse amplitude, pulse frequency and the on/off percentage of time during the pulse frequency period (otherwise known as the duty cycle). In addition, the composition and viscosity of the surrounding electrolyte fluid, e.g. body fluids such as interstitial fluid, cerebrospinal fluid, etc., as well as the electrode material and current density influence the nature and extent of the formation of electrolysis by-products.

In a preferred embodiment of the invention, plusatile DC currents are utilized to minimize possible deleterious products. In this embodiment of the invention, the pulse frequency is generally between 0.1 Hz and 1000 Hz, the duty cycle is generally between 0.1% and 10% and the current density is generally between 0.01 mA/cm2 and 100 mA/cm2. However, the broader scope of this invention is not intended to be limited by these embodiment and conditions. It is noted that other conditions, materials and structures may be employed such as those described by in the following sections that permit wider current limits and parameters, including continuous application of direct current.

pH—Electrophoretic activity may result in the electrolysis of water, forming either acid or base in the vicinity of the electrode (typically acid, H⁺, at the anode and base, OH⁻, at the cathode). In certain situations, the generated base or acid may overwhelm the surrounding fluid's buffering capacity, substantially altering the local pH and potentially adversely affecting the surrounding tissues and cells. One embodiment to ameliorate this generation of acid or base is to employ a modified form of electrophoresis whereby the polarity of the electrodes is reversed periodically. That is, although electrophoresis is substantially DC in nature, by altering the polarity of the electrodes intermittently, an electrode which had been the site of acid generation now becomes a source of base generation, and vice versa. This switching of polarity, if performed with the appropriate periodicity, will substantially eliminate adverse pH effects yet will have minimal effects upon the net migration of the charged species, e.g. the polarity reversal is for such a short period that the charged biomolecules do not migrate substantially back to their initial location. In one embodiment of this invention, the polarity is reversed in an asymmetric fashion, such as by time of pulse period or by current amplitude, to achieve neutralization of generated acids or bases.

An alternate embodiment of the invention is to provide additional buffering materials or compounds either as part of the structure or as delivered solutions. That is, the structure of the device may be composed of materials which function in part as a binder to the acid/base such that the acid or base generated is immediately bound to the material, thereby neutralizing these reactive species. Such materials may include structural carbonates or coatings of ion exchange resins. Alternatively, solutions may be supplied which have additional buffering capacity, adjusted to physiological pH such that the generated acid or base will be adsorbed by this additional buffering capacity. This method may be used alone or in combination with the alternating polarity mentioned above to negate the effects of generated acid or base.

However, it may be in some circumstances that an altered pH in the surrounding tissue may be beneficial to maintaining device function, e.g. aiding the breaking of ionic bonds, salt bridges or weak covalent bonds of surrounding structures, etc. In such embodiments of the invention, the need to buffer generated acid or base would be unnecessary or otherwise qualified and the generation of either acid or base a desired outcome.

Electrolysis Zone—The process of electrolysis or breaking down of water molecules creates a highly reactive zone extending from the surface of the electrode into the overlaying space, up to several hundred nanometers, dependent upon, among other factors, the structure of the electrode, the electrode potential and the composition of the solution. This zone may be harmful to the surrounding tissue directly or the process of electrolysis induces a rejection response through the formation of radicals which generate antigenic species. In one form of the invention, the electrodes may have one or electrically active surfaces positioned of the electrode surface away from the surrounding tissue at a sufficient distance to mitigate the effects of electrolysis, e.g. a distance generally greater than 1 micron, and thereby segregating the tissue from this highly destructive environment. In one embodiment of this form of the invention, the electrodes are physically separated from the tissue by an overlying semipermeable structure or gel. A semipermeable structure in the context of this invention is a structure, membrane, mesh or gel, which provides fluid and small molecule access to the electrode surface while physically distancing the electrode from contact with surrounding tissue. Therefore the dimensionality of the pores of such a structure should be less than the dimensionality of the surrounding cells and tissues. In general, this indicates a pore size that is less than 10 microns in diameter is desirable. In alternate embodiments of the invention larger pore dimensions is offset by increased fluid path length or tortuousity thereby permitting the use of meshes or polymers with pore sizes considerably larger in diameter, e.g. 1 mm.

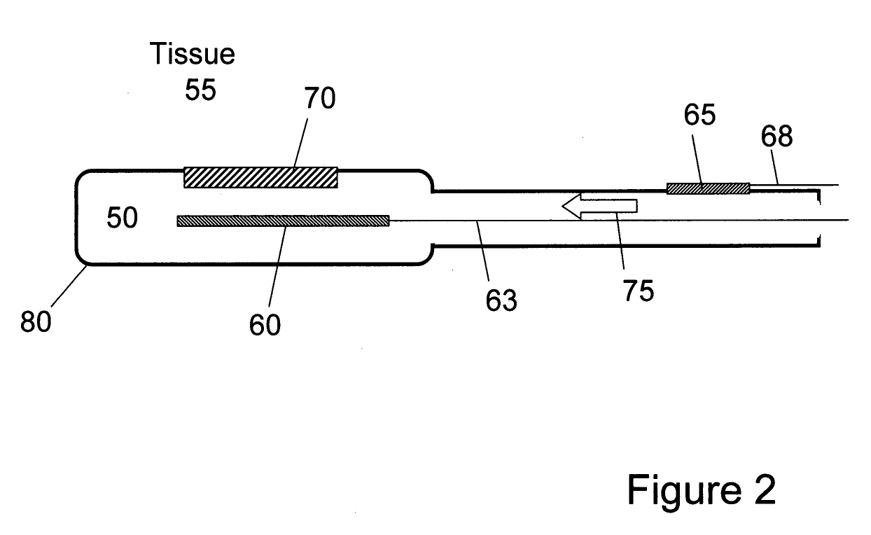

This semipermeable structure may also serve as the division between the interior or lumenal space of the device and the exterior of the device. In yet other embodiments of this invention, the semipermeable structure may provide addition roles within the device, e.g. as a means or site of fluid delivery (FIG. 2) or as a mechanical structure providing a support for surrounding tissue ingrowth and neovascularization.

In FIG. 2, one end of a fluid delivery device (80), e.g. a catheter like device, is shown having a lumenal space (50). Positioned within this lumenal space and beneath a semipermeable structure (70) is a first electrode (60). This first electrode is connected to a power supply/control unit by means of an insulated wire (63). On the outer aspect of the device, is a second electrode (65), likewise connected to the same power/control unit as the first electrode by means of an insulated wire (68). Upon activation of the electrodes, the electrical current passes from the surface of the first electrode (60) into fluid present in the lumenal space, traverses through the semipermeable structure (70), through the surrounding tissue (55) and completes the circuit at the second electrode (65). It should be noted that no orientation or polarity of activation is implied by this description of the electrical pathway. The semipermeable structure also serves as the site of fluid delivery from the interior lumenal space of the device, as indicated by the arrow (75). Fluid passing down the lumenal space of the catheter will exit from the device (80) through the semipermeable structure (70) in pass into the surrounding tissue (55).

Alternative embodiments may include but are not limited to the positioning of the electrodes within structures, e.g. narrow channels or grooves, on the exterior surface of the device. Alternatively, the electrodes may be positioned within the device so that the surrounding tissues or cells are not in direct contact with the electrolysis zone.

Gas Generation—Another by-product of electrophoresis is gas generation at the electrodes. In aqueous solutions, the positively biased anode typically generates oxygen while the negatively biased cathode typically generates hydrogen. The amount of gas generated is dependent upon the current utilized. If the rate of evolution is sufficiently low per unit area, then the generated gas will dissolve into the surrounding fluid without bubble formation (this is dependent, among other factors, upon the rate of electrolysis per unit area, electrode composition, surface roughness of the electrode, etc.). However, if higher currents are required in order to minimize the body's rejection response, the overall electrode dimension, shape and number of electrodes may be altered to accommodate higher currents necessary to mobilize the biomolecules while avoiding bubble formation. Therefore, in one embodiment of the invention, gas bubble formation is minimized by enlarging the electrode surface area relative to the current employed in order to facilitate diffusion of the gas into the surrounding fluid. Such enlargement of surface area also may benefit charge transfer characteristics of the electrode, in general. It should be noted that, in certain circumstances, it may be that the gas generation, particularly oxygen, may provide a benefit to the surrounding tissue and therefore electrolysis may be employed for this reason, e.g. U.S. Pat. Nos. 6,368,592, and 5,788,682. In one embodiment of the invention, therefore, the generation of gas is a desired outcome in addition to the use of electric fields to minimize capsule formation.

An alternate embodiment by which to minimize gas bubble formation is to employ agents to absorb the gas as it is generated. This may be accomplished using materials which are employed also as electrodes. This is the case with certain metals, e.g. titanium or platinum at positively biased electrode (anode) which form oxides in the presence of the generated oxygen or palladium at the negatively biased electrode (cathode) which absorbs hydrogen. Alternatively, these materials may be located near to the electrodes but not necessarily serving as the electrode, e.g. a mesh or structure overlaying the electrode which absorbs the gas in question.

A third method approach to resolving evolution of gas and subsequent bubbling is the electrode or structure associated with the electrode being a semi-permeable structure in contact with body fluids on one side and providing an escape or sequestration chamber for the generated gas on the other. Such a structure, e.g. a membrane, mesh or brush-like structure, which allows passage of the generated gas on one side and current through the fluid on the other. The gas would either vent to outside of the body via a conduit means or be sequestered in a reservoir. This reservoir may contain additional agents or materials to absorb the generated gas, thereby reducing the volume and pressures. A further refinement of this embodiment is that the mesh or membrane structure also contains additional agents, e.g. ion exchange materials, to sequester additional by-products of electrolysis specifically the generated acid or base.

Electrodes

The electrophoretic circuit may be completed using a one or more electrodes of various geometries and composition. In a preferred embodiment, there is a least one first electrode comprising at least part of or in close proximity to a critical structure upon a device and at least one second electrode or second electrode located in a region non-critical for device function. The electrolytes and fluid provided by the surrounding tissue providing a means of electrical connection between the first and second electrodes. In certain embodiments of the invention, the second electrode may be placed on the outer aspect of the subject's skin or body. In such embodiments, a means to ensure electrical contact, e.g. saline solution or conductive gel, should generally be present to provide electrical contact from the second electrode to subdermal regions.

In the context of this invention, close proximity indicates a distance generally in the range of immediate contact between the electrode and the critical feature, e.g. the first electrode may comprises a part of the critical structure of the device, or close proximity may extend to a distance of several centimeters separation between the first electrode and the device critical feature or structure. In such circumstances, in addition to factors such as current amplitude, and device geometry, the electrical path through the tissue will greatly influence the distance or spacing between the first electrode and critical feature or structure.

In alternate embodiments of the invention, a plurality of first and second electrodes are utilized. In these embodiments, the activation of these electrodes may be in defined sequences or order involving one or more electrodes of a specific bias at any point in time in order to facilitate the mobilization of the biomaterials. In one form of this alternate embodiment, a series of electrodes are positioned as concentric bands around a tubular device, e.g. a catheter. By sequential activation of sets of these electrodes in a wave-like pattern, charged materials or cell types may be “walked” away from critical features, e.g. fluid infusion sites, towards non-critical locations upon the device. This operation may be repeatedly applied in order to facilitate this movement of materials. Variations of such electrode embodiments include sequential activation of one or more sets of electrodes over a period time, e.g. days or weeks, to control fibrous capsule formation upon the device attributable to either continued inflammatory activity or mechanical disruption. In yet other alternate embodiments, similarly biased electrodes may be placed at the two or more critical features of the device and the counter electrode placed elsewhere. This arrangement serves to diminish rejection at multiple points simultaneously, thereby improving overall device performance.

Electrodes may be constructed from conductive or semiconductive materials including, but not limited to: metals such as gold, platinum, palladium, silver or titanium; organic conductive polymers; conductive gels or epoxies such as silver impregnated pastes; graphite; carbon or mixed composition nanotubes; doped silicons or other semiconductive structures; or layered/mixed form structures comprised of inert and conductive materials, such as structures fabricated using MEMS-like (MicroElectroMechanical Systems-like) processes or techniques, e.g. micromachined constructs having metallic layers or sections upon high resistivity substrates. In general, a preferred material to be used for the composition of the electrodes is platinum or platinum alloys.

The electrodes may be patterned on or affixed to either interior aspects or exterior aspects of the device or separated from the device or critical structure. In a one embodiment of a device utilizing the apparatus of this invention, the first electrode is built into the structure of a critical feature, e.g. a fluid access site, of a device while the second electrode is located elsewhere on the device. In another embodiment of the invention, the first electrodes are located in proximity of one or more critical locations on a device but do not form a part of the device structure. That is, one or more electrodes may be positioned in close proximity to the critical features of the device (but not on the device) thereby providing the necessary electrophoretic activity to mobilizes the biomolecules/cells associated with clogging or rejection away from these critical features or structures of the device.

In yet other alternate embodiments, the first electrode is on the structure of device while the second electrode is located upon a second introduced structure or material, e.g. an introduced second electrode or a second device having, as part of its function, the second electrode. Such systems require the completion of the circuit between the first and second electrodes by external or other form of electrical connection in addition to the electrical pathway provided by tissue conductivity. In other embodiments, neither the first nor second electrodes are part of the structure of the device but are positioned in close proximity to the device and the device critical features.

In a preferred embodiment of the invention, the electrode form typically is primarily planar, having one surface exposed to the surrounding medium and the other surface supported by underlying structures, e.g. the outside wall of the tube or catheter. However, other embodiments of electrodes are conceivable. These embodiments include, but are not limited to, electrode structures that are either: predominantly conical in shape; brush-like in composition, e.g. arrayed nanotubes; transitory (formed from conductive fluid droplets akin to those used in dropping mercury electrodes which provide fresh surfaces periodically); or are formed from wires or other conductive materials extending along edges of fabricated surfaces.

In yet other embodiments of the invention, one or more electrodes may be located within lumenal spaces of devices or otherwise separated from direct contact with surrounding tissue while still in electrical contact by fluidic means to said tissue. Such means of separation include but are not limited to, coating of the electrode surface with permeable gels such as polyethylene glycol, or polyurethane, or employing meshes, membranes or other structures, e.g. glass frits, such that the electrode surface is not in direct contact with surrounding cells, membranes or extracellular structures. Such methods allow the application of the current while distancing the surrounding tissue from the destructive electrolysis zone.

Control Circuitry

Activation of electrodes for the purpose of electrophoresis may be done in a variety of fashions, including, but not limited to, activation upon command, activation periodically, or being activated substantially continuous fashion, e.g. always “on”. That is, circumstances may indicate that a defined pattern of activation, followed by lesser activity. An example of this embodiment is the use of frequent pulses of electrical current for the immediate period post implantation of the device, e.g. 24-72 hours, to limit the initial steps of the wound healing cascade followed by a lower frequency or periodicity of application for the remainder of the implant's lifetime to deal with a lower, more chronic inflammatory activity.

In alternate embodiments of the invention, additional activation, upon demand, may facilitate removal of additional debris occluding fluid transport across the access port. Activation of the electrodes would then be upon set by therapeutic agent delivery or sensor needs so that occlusion was minimized during or just prior to therapeutic administration or sensor activation. Thus, a variety of activation schemes and profiles are possible within the scope of this invention and this invention is not limited to the embodiments described.

Control and power of the electrophoresis process may be accomplished with devices as simple as a battery plus microcontroller or as complicated as an external power circuit plugged into a wall plug plus controlling software. The needs, cost and lifetime of the implanted device will govern the form of power supply and control used. In the case of fully implanted devices, power may also be supplied using inductive or other power coupling means in addition to on-board batteries or other forms of power storage. Power sources may also include indirect means, e.g. utilizing an inductively coupled means to transmit energy to the electrical circuit or by use of energy obtained from mechanical or chemical activities.

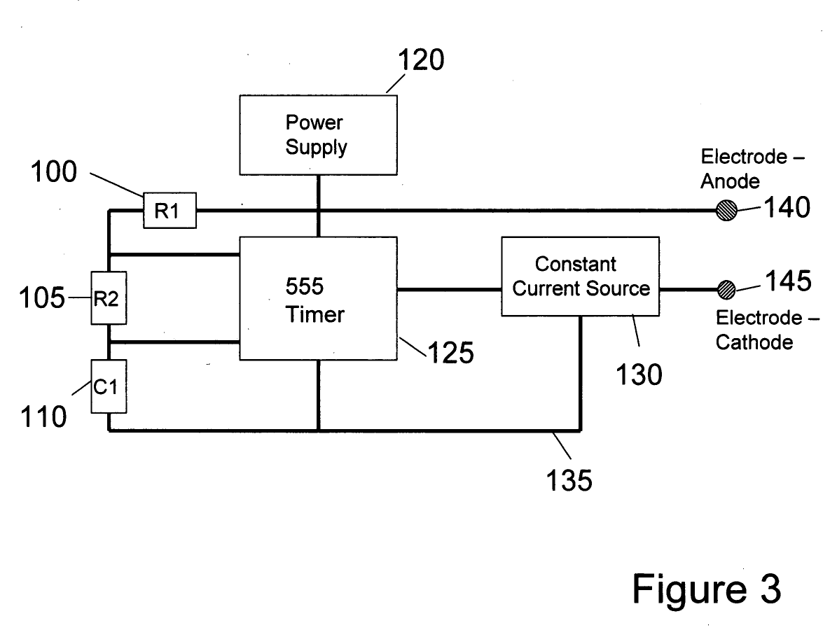

FIG. 3 illustrates the components of one such circuit for the controlled delivery of pulsatile DC currents to electrodes. One skilled in the art of electronics will recognize that numerous other circuits that accomplish this purpose are conceivable and are covered within the scope of this invention. Power is supplied by the Power Supply (120), typically a battery. The repetitive pulse is generated within the 555 timer (125) (an industry standard integrated circuit available from Texas Instruments, Philips Electronics, National Semiconductor, etc.). Frequency and duty cycle are determined by external resistors, R1 (100), R2 (105) and capacitor, C1 (110). The output of the timer drives a constant current source which in turn, provides the constant current source (130) through the circuitry (135) to the anode electrode (140) and current sink to the cathode electrode (145).

An example calculation for determining duty cycle employing the circuitry of FIG. 3 is shown in Equation 1:

Duty cycle (Ratio of ON time to OFF time)=R2/(R1+2 R2) Equation 1)

Assuming R1=98 Kohm and R2=1 Kohm and C=10 uf, then the duty cycle equals 1/(98+2*1) or 1% and the pulse frequency equals 1.44 Hz. One skilled in the art of electronics will readily appreciate that more complex circuits, involving delays, changes of pulse amplitudes or frequencies as well as additional variety of pulse patterns may be readily conceived and employed within the scope of this invention.

In one embodiment of the invention, regulation of the control circuitry, i.e. the programming of the amplitude and periodicity of the current to be delivered, is set prior to installation of the invention into a medical device. In another embodiment of the invention; a separate means to adjust or provide control electrical current output post-installation is provided. Such means include, but are not limited to, keypad entry, wireless control, or by optical or acoustic means.

Other embodiments of the invention providing for adjustment/activation of the currents applied also include the use of input or controls provided within a larger medical device or system employing this invention. In yet other embodiments of the invention, feedback from sensors indicating the need to alter the current profile, either associated with the apparatus of this invention or as part of other devices, may be sent to a control circuit in an automatic fashion and thereby providing a “closed-loop” system of operation of the apparatus of this invention within the body of a subject.

Devices

The novel use of apparatus to produce electrical currents to retard or diminish fibrous capsule formation is suitable for use with a variety of implanted medical devices. These devices and systems include, but are not limited to, catheters, MEMS-based drug delivery systems and removable/replaceable diagnostic or drug delivery devices. In addition, the invention may be useful for systems such as that described in U.S. patent application Ser. No. 10/032,765 “Gateway Platform for Biological Monitoring and Delivery of Therapeutic Compounds”, incorporated by reference in its entirety herein, which describes devices suitable for percutaneous drug delivery and sampling of interstitial fluids. The use of the structure of this invention may also be applied to other devices and systems which may benefit from reduction of the wound healing response or encapsulation control or having use of the electric fields generated in the surrounding tissue.

For instance, application of the electric currents in conjunction with a subdermal therapeutic delivery means may result in accelerated dispersal of the therapeutic agent through surrounding tissues. This is, if the therapeutic agent possesses a net charge, it will migrate along the electrophoretic path into the surrounding biofluid, in accordance with its net charge, mass, effective field strength, etc. It should be noted that this process differs from use of electrophoresis as means of delivery of charged materials from the interior lumen of devices to the exterior space with devices, e.g. the technique of iontophoresis. In these inventions, the electrophoretic path is substantially between interior space and the exterior passing through a semipermeable structure and electrophoresis is typically employed to mobilize the therapeutic agent from an interior reservoir or site through the semipermeable membrane. Thus, this accelerated dispersal of drugs or agents through surrounding tissue by electrophoretic means represents a novel aspect of one embodiment of this invention.

General operation of the apparatus of this invention, including first and second electrodes, power supply and control circuitry, requires the installation of the apparatus into the body of a subject. Such installation is preferably done in coordination with the installation of a medical device. Such installation may be concurrent with the implantation of the medical device, e.g. the apparatus forms a portion of the device, or the apparatus may be installed at a time other than that of the medical device installation, e.g. to permit post-surgical recovery following installation of the medical device. Once installed, the apparatus of the invention may be activated either upon command, e.g. manually activation of a switch, or by instruction, e.g. remote wireless instruction. Upon activation, the electrical current is passed through the tissue from one or more first electrodes to one or more second electrodes. The nature of this electrical current, including the amplitude, periodicity, frequency, duty cycle, and polarity may be based upon the instructions supplied by the control circuitry or as part of the construction of the apparatus itself, e.g. the polarity being set by battery contact orientation. Further control of the apparatus, including the cessation of activity, may be accomplished in a variety of fashions, including but not limited to, manual command, pre-set programming or received instructions.

The following embodiments are representative to the types of devices possibly employing the use the apparatus of this invention to minimize encapsulation. One skilled in the art will readily recognize that additional devices and systems are conceivable and that the scope of this invention is not limited to those embodiments shown below.

Percutaneous Catheter—A typical use for electrophoretic control of the body's rejection response is to reduce rejection of implanted catheters which continuously or intermittently deliver therapeutics or fluids over an extended period of time, e.g. days or weeks, to the body. Sites for these deliveries include but are not limited to, subcutaneous, cerebrospinal, targeted organ or intraperitoneal locations.

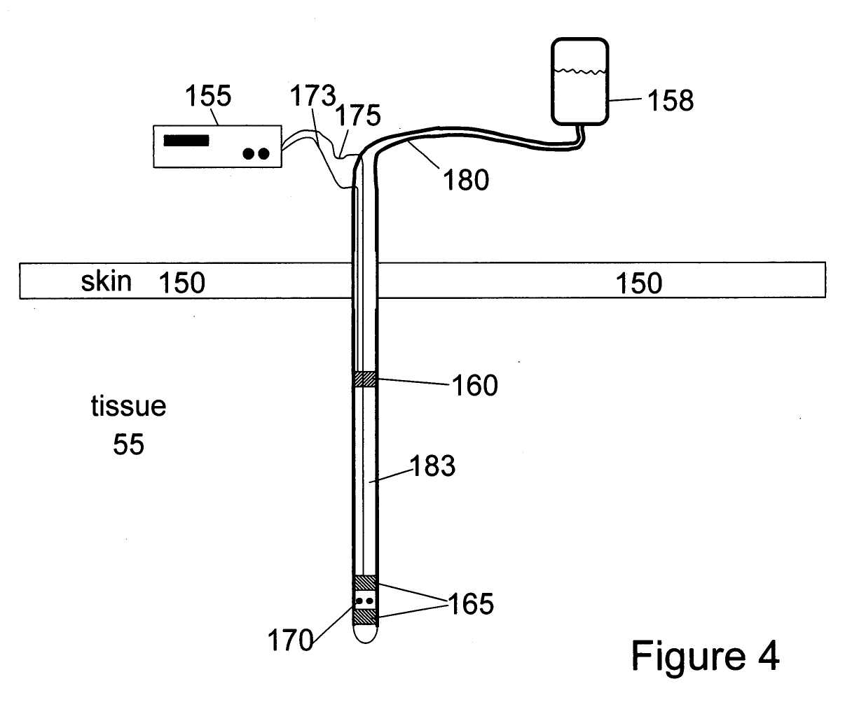

Such a percutaneous catheter-like device is shown diagrammatically in FIG. 4. In this figure, the catheter-like device (180) has a fluid reservoir (158) for the parenteral delivery or infusion of therapeutics, fluids or drugs. Located on the outer aspect of the catheter beneath the skin (150) are a set of first electrodes (165) and a second electrode (160). In this embodiment of the invention, the electrical current supplied by an external power/control unit (155) passes through insulated wires 175 and 173, to electrodes (165) and (160). The current passes from the first electrodes (165) to the second electrode (160) through the surrounding tissue (55). In alternate embodiments of the invention, such power supply/control circuitry are located within the device and may be incorporated into sections of systems implanted into the body of the subject.

Also shown in FIG. 4 is the lumenal space (183) of the catheter-like device through which the fluids/therapeutic agents pass. The agents exit from the lumenal space by holes (170) adjacent to the first set of electrodes. The proximity between the holes (170) and the first electrodes (165) aids in the efficiency of maintaining patency and reducing fibrous capsule formation in this region. Alternatively, a catheter-like device such as that shown in FIG. 4 may employ a polymeric mesh through which the therapeutic solution passes into the surrounding interstitial fluid, instead of the holes (170) indicated.

Driving the current through the electrodes may be through a circuit such as previously illustrated in FIG. 3 or by an external control unit connected to a power supply. Suitable power supplies are readily available off-the-shelf, e.g. from Keithley Instruments. In one embodiment of the invention, the first electrodes are biased to serve as anodes. As an example of one possible embodiment of applied electric fields which may be transmitted through such a device, a weak current (approx. 1 uA) is passed for 10 sec once every 2 minutes from the first electrodes to the second electrodes.

A commonly considered side product of electrolysis is altered local pH. Based upon 1 A=6.24×10(18) electrons per sec and assuming each electron represents either the formation of 1 molecule of base or acid, then activation of the electrode for 10 seconds will generate approximately 1×10(−10) moles of acid and base. Using a simple model to predict the migration of the acid or base and assuming diffusion constant for a small molecule to be approximately 3×10(−6) cm2/sec, then, during the time delay between electrode activations, 110 seconds, the generated acid or base will migrate approximately 0.25 mm (in any one direction). The volume described by this migration for a single 1 mm electrode band around a 3 mm circumference catheter is approximately 0.75 ul leading to an average acid or base concentration within this volume of 133 uM.

The buffering capacity of the surrounding fluid extracellular fluid varies upon the composition of that fluid, i.e. its protein and modified proteins, ions, etc., and therefore will vary between blood, interstitial fluid, or cerebrospinal fluid. If the buffering capacity of serum is assumed to be primarily set by bicarbonate/carbonate ions, and the concentration of this buffer is approximately 24 mM (at pH 7.4 with a pKa of 7.6). Assuming these small ions readily equilibrate between interstitial fluid and serum, then the interstitial fluid has approximately two orders of magnitude excess buffering capacity. Thus, based upon this electrode activation protocol, the local generation of acid and base will not grossly alter the pH of the surrounding interstitial fluid, <0.01 pH unit, and therefore, the generated acid/base should not be detrimental to the surrounding cells and tissue using this electrical current protocol. Other electrode designs, currents, frequencies, periodicities and protocols may be employed which do not exceed local buffering capacity and this invention is not limited to those conditions supplied in the above example. As noted earlier, if higher currents which alter pH are employed and these are determined to be detrimental, then other means, e.g. materials, alternating currents, may be employed to reduce the extent of pH change, if necessary.

Likewise, it can be shown that a 1 uA current for 10 seconds generates approximately 1.6×10(−6) mg of oxygen per 10 seconds, leading to a local concentration (within 0.75 ul) of 2.13 mg/l, with similar levels for hydrogen. At 37° C., oxygen saturation is 7 ppm (mg/l), therefore, bubble formation may not be observed using this current and electrode geometry. Local micronucleation of bubbles may occur on the surface of the electrodes, however, these should absorbed into the surrounding fluid.

Detrimental effects within the electrolysis zone, i.e. the electrode surface, may necessitate coating the electrodes with a semipermeable mesh to separate the electrodes from the surrounding cells. One means to accomplish this was shown in FIG. 2 where the electrode was positioned within the lumen of catheter-like device. An alternative means to accomplish this is by coating the electrode surface with a non-reactive, biocompatible pourous layer, e.g. a polymer hydrogel, (such as a urea/polyethyleneglycol gel or polyacrylamide gel), or by covering the electrode surface with a defined, non-conductive structure such as a thin dacron membrane.

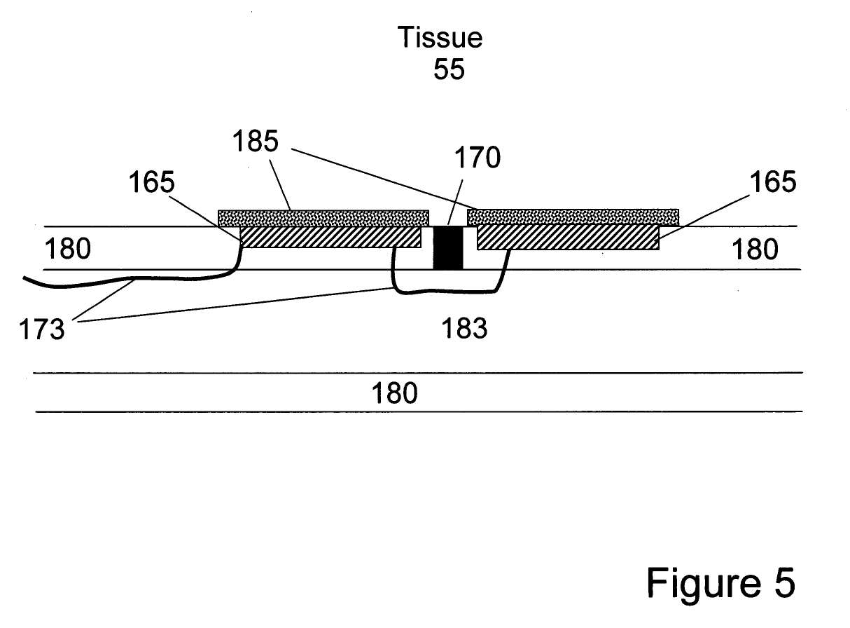

One such modification by coating of the electrode surface is shown in the cross-sectional view provided by FIG. 5. In this figure, a first set of electrodes (165) are shown located on the outer aspects of a portion of a catheter-like device (180). Fluid access from the lumen of the catheter-like device (183) to the surrounding tissue (55) is provided by one or more holes (170). Connection of the electrodes to the power supply/control circuit is via insulated wires (173). In this embodiment of the invention, the electrodes (165) are directly coated with a hydrogel-like material (185) to provide fluid access yet maintain a distance between the surrounding tissue (55) and the outer aspects of the electrode surface.

The catheter-like device shown in FIG. 4 presents one means by which to employ the method and apparatus of this invention to reduce encapsulation and thereby improve patency of a catheter-like device. Alternate devices, used for therapeutic delivery or for sampling of biofluids for analytes, employing a variety of electrode geometries, materials and current protocols are readily conceivable and this example is not intended to limit the scope of this invention.

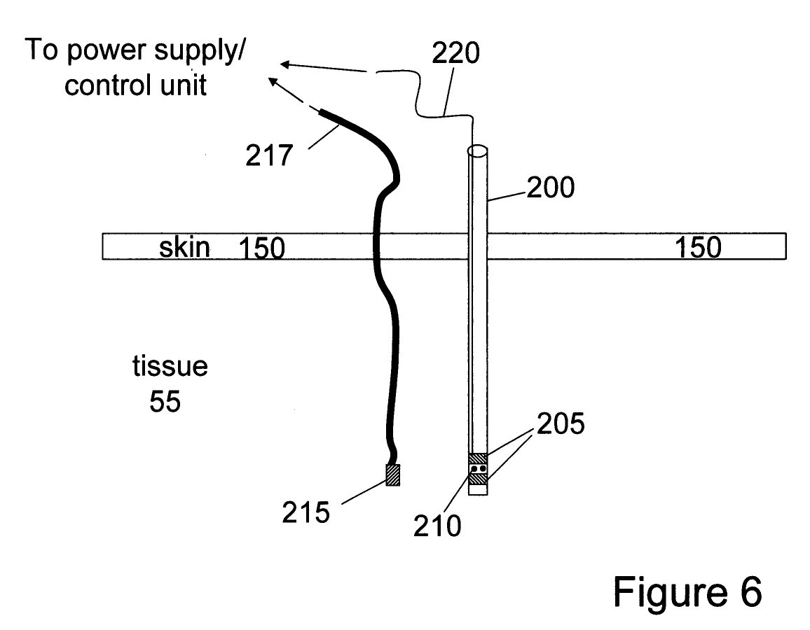

Percutaneous Catheter Having Separate Second Electrode—An example of a system employing a removable/replaceable electrode is shown diagrammatically in FIG. 6. In this example, a catheter system similar to FIG. 4 is utilized, with modification. That is, electrical connections (217 and 220) enable control and electrical currents to be supplied to the electrodes (205 and 215). The percutaneous catheter-like device (200) traverses through the skin (150) and into the underlying tissue (55). This device may serve as a means of infusing fluid or therapeutic agents through the holes located near the end of the device (210). However, only the first electrodes (205) are located on the outer aspects of the catheter-like device. The second electrode (215) is removed from the structure of the catheter and is connected via a flexible insulated wire (217) to the power supply/control unit. This second electrode may be constructed from a variety of materials. In one example of this embodiment of the invention, the electrode may be constructed of platinum approximately 1 mm long connected to polyurethane coated copper wire (approximately 26 gauge). This electrode may inserted subcutaneously using a trocar-like device and the electrode positioned to be near, e.g. within several centimeters, of the first electrodes.

In one embodiment of the invention, as shown in FIG. 6, the currents employed and times of activation may be the same as those utilized in the apparatus presented in FIG. 4, dependent upon factors including, but not limited to, the geometry of insertion, device geometry, tissue characteristics and apparatus structure and materials. One difference to this system as opposed to that presented in FIG. 4 is that any fouling, degradation of performance or accumulation of rejection response products observed at the second (counter) electrode is resolved by removal of this electrode and replacement with a new electrode. Thus, long term rejection/diminished performance of the second electrode may be resolved by replacing this electrode periodically. Extensions of this embodiment of the invention include the use of a plurality of counter electrodes simultaneously to provide more equivalent electrophoretic fields than a single electrode would provide.

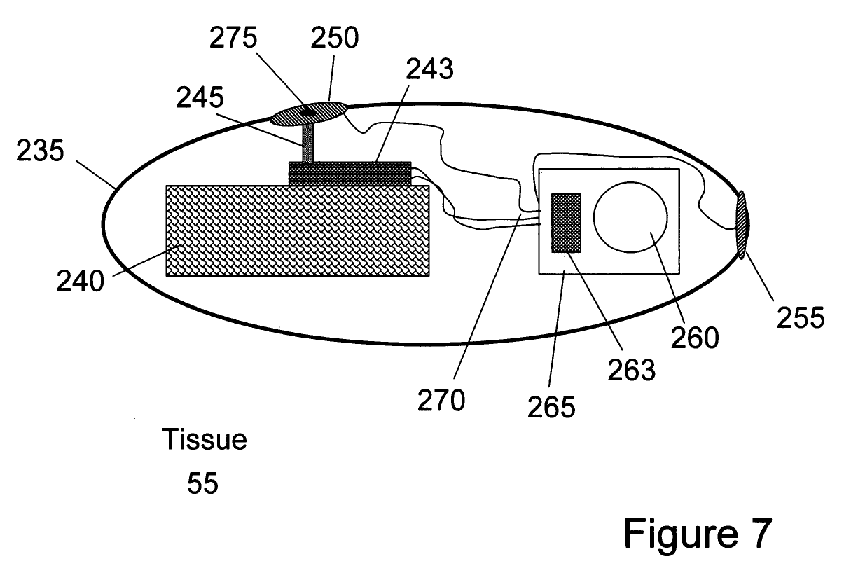

Fully Implanted System—An entirely subcutaneous implanted device designed to deliver therapeutics and/or sampling of biofluids for analytes is shown in FIG. 7. This device differs from the percutaneous devices presented in FIGS. 4 & 6 in that the entire device is fully located beneath the skin and therefore does not directly utilize an external power supply or control circuitry to activate the electrodes.

As shown in the cross sectional view provided by FIG. 7, a means for drug delivery is shown schematically within the body of the device (235). That is, the device contains a reservoir (240), pump unit (243) and conduit (245) through which the therapeutic agents may dispensed into the surrounding tissue (55) through one or more holes (275). Control and power for the delivery activity is provided by a battery (260), an integrated circuit (263), additional circuitry (265) and wiring/circuitry (270). One skilled in the art of electronics will readily appreciate that such integrated circuitry (263) plus power (260) may be also utilized to drive an electrical current from one or more first electrodes (250) to one or more second electrodes (255). In adapting this device for sampling, the function of the pump plus reservoir is replaced by appropriate sensors and signal amplifiers, however, the use of integrated circuitry plus power remains.

In the diagrammatic example shown in FIG. 7, a battery supplies the necessary power. In alternate embodiments, inductive power or other sources of power may be employed. Assuming 1 uA pulsatile current (10 sec “on” during every 2 minutes), the device is active approximately 8% of the time. A typical small coin cell battery 1 cm×0.3 cm in size has approximately 20 mA hours lifetime at 3 V. At a constant (not pulsatile) 1 uA level of drain, this battery would last for over 20,000 hours (or approximately 2 years). Therefore, a variety of fully implanted systems and devices employing electrophoresis as a means of limiting rejection are feasible, based upon device needs and apparatus requirements.

One will readily recognize that other devices, current protocols, electrode geometries, power sources, and control circuitry, etc. are readily conceivable and this methods and apparatus of this invention are not limited to the embodiments shown herein.

We claim:

1. A method using electric fields to reduce the body's rejection response to materials, devices or systems placed in part or in whole subdermally consisting of:
a. one or more first electrodes subdermally located in the close proximity of a critical structure or feature of a device introduced into the body;
b. one or more second electrodes located elsewhere;
c. and the passage of an electrical current through tissue between the first set of electrodes to the second set of electrodes for the purpose of minimizing encapsulation in the area of the critical structure or features of the device.

2. The method of claim 1 wherein the electrical current is substantially DC in nature.

3. The method of claim 1 wherein the electrical current is substantially pulsatile in nature.

4. The method of claim 1 wherein the current density of one or more first electrodes is generally between 0.01 mA/cm² and 100 mA/cm².

5. The method of claim 1 wherein the method includes the use of a semipermeable structure to separate one or more first electrodes from surrounding tissue.

6. The method of claim 1 wherein the method includes the use of a semipermeable structure to separate one or more second electrodes from surrounding tissue.

7. A method using electric fields to reduce the body's rejection response to materials, devices or systems placed in part or in whole subdermally consisting of:
a. one or more first electrodes subdermally located in the close proximity of a critical structure or feature of a device introduced into the body;
b. one or more second electrodes located elsewhere;
c. and the passage of a pulsatile electrical current through tissue between the first set of electrodes to the second set of electrodes for the purpose of minimizing encapsulation in the area of the critical structure or features of the device.

8. The method of claim 7 wherein the current density of one or more first electrodes is generally between 0.01 mA/cm² and 100 mA/cm².

9. The method of claim 7 wherein the method includes the use of a semipermeable structure to separate one or more first electrodes from surrounding tissue.

10. The method of claim 7 wherein the method includes the use of a semipermeable structure to separate one or more second electrodes from surrounding tissue.

11. An apparatus for the delivery of electric fields to reduce the body's rejection response to devices placed in part or in whole subdermally having:
a. one or more first electrodes subdermally located in the close proximity of a critical structure or feature of a device introduced into the body;
b. one or more second electrodes located elsewhere;
c. control circuitry and power supply to provide for the passage of an electrical current through tissue between the first set of electrodes to the second set of electrodes for the purpose of minimizing encapsulation in the area of the critical structure or features of the device.

12. The apparatus of claim 11 wherein one or more first electrodes is affixed to the device.

13. The apparatus of claim 11 wherein one or more second electrodes is affixed to the device.

14. The apparatus of claim 11 wherein one or more first electrodes is not affixed to the device.

15. The apparatus of claim 11 wherein one or more second electrodes is not affixed to the device.

16. The apparatus of claim 11 wherein the device is percutaneous in nature.

17. The apparatus of claim 11 wherein the device is fully implanted.

18. The apparatus of claim 11 wherein one or more first electrodes is separated from tissue by a semipermeable structure.

19. The apparatus of claim 11 wherein the device is used for the purpose of therapeutic agent delivery.

20. The apparatus of claim 11 wherein the device is used for the purpose of sampling of biofluids for analytes.
